(19) United States
(12) Patent Application Publication
(10) Pub. No.: US 2010/0160869 A1
(43) Pub. Date: Jun. 24, 2010

(54) Medical Needle Assemblies

(75) Inventor: Barry Peter Liversidge, Colchester (GB)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: Barry Peter Liversidge, Colchester (GB)

(21) Appl. No.: 12/714,169

(22) Filed: Feb. 26, 2010

Related U.S. Application Data
(63) Continuation-in-part of application No. 10/518,950, filed on Jun. 29, 2005, now Pat. No. 7,699,813.

(30) Foreign Application Priority Data
Jun. 22, 2002 (GB) 02 14452.5
Feb. 3, 2003 (GB) 03 02393.4

(51) Int. Cl. A61M 5/32

(52) U.S. Cl. 604/198

(57) ABSTRACT

A safety arrangement for a medical needle 15 has a support 14 for carrying the mount end of the needle, and a sleeve 16 slidably mounted on the support 14 for movement from an initial position to a retracted position and then back to a protecting position where the sleeve 16 covers the tip 21 of the needle 15. A spring 32 urges the sleeve towards its protecting position and a blocking member 23 projects forwardly from the support 14 and is movable between non-blocking and blocking positions. In the non-blocking position the sleeve 16 is free to slide from its initial position to its retracted position. On movement of the sleeve to its protecting position, the blocking member 23 moves to lie between the support 14 and sleeve 16 to prevent subsequent movement of the sleeve away from its protecting position. A control member 27 for the blocking member 23 is slidable within the sleeve from a first position at the rearward end of the sleeve to a second position forwardly therefrom. The sleeve is translucent or transparent and the control member is of a strongly coloured material so that when the arrangement has been used, the control member is clearly visible within the sleeve to indicate the device has been used and the sleeve is locked against rearward movement.

### CROSS REFERENCE TO RELATED APPLICATIONS

This application is a continuation-in-part of U.S. patent application Ser. No. 10/518,950 filed on Jun. 29, 2005 which is based on International Patent Application No. PCT/GB2003/002689 filed on Jun. 23, 2003, which in turn claims the priority of UK Patent Application Nos. 0214452.5 and 0302393.4, filed respectively on Jun. 22, 2002 and Feb. 3, 2003, though only the priority of UK Patent Application No. 0214452.5 is claimed in this continuation application. All of these said applications stand in the name of Barry Peter Liversidge.

### BACKGROUND OF THE INVENTION

a) Field of the Invention

This invention relates to a safety arrangement for a medical needle having a mount end and a sharp tip, intended for penetration of a human or animal body, or for other medical uses such as the penetration of a pierceable membrane of an intravenous medication system. The invention further relates to a safety arrangement including a medical needle as aforesaid, ready for use and also to a medical syringe or a medical injector, provided with the safety arrangement. For convenience, in the following all medical uses of the safety arrangement will be described simply as the penetration of a body, even though specific embodiments may be intended for other medical uses.

b) Description of the Related Art

Fluids of various kinds may be administered to a human or animal body by means of a hollow needle in conjunction with a source of the required fluid. For example, such a needle may be used in conjunction with a syringe holding a liquid drug, the needle being used to penetrate the body at the site at which the drug is to be received. Equally, body fluids may be withdrawn by using a hollow needle which is used to penetrate the body until the tip is located at the site from which fluid is to be withdrawn.

A recognised hazard for clinicians and other persons using medical needles for the above described purposes is the risk of a so-called needle-stick injury—that is to say the accidental penetration of the clinician's skin by the needle. Prior to the use of the needle to supply a fluid to or to withdraw fluid from a body, this rarely presents much of a problem, though once the needle has been used on a body, there is a very much higher risk of a serious consequence for the clinician. During use of the needle to penetrate the body tissues of a patient, the needle is likely to become contaminated with various organisms and should a needle-stick injury occur, these could infect the clinician.

There have been numerous proposals for protecting the sharp tip of a used needle, in order to reduce the risk of a needle-stick injury following use of the needle. Some proposals have actually increased the likelihood of such an injury by virtue of the action which must be performed to protect the tip, even if the risk thereafter is, lessened. Despite all of the proposals which have previously been made, very few have achieved commercial success, nor has there been wide acceptance by the medical industry. Many proposals are somewhat complex and involve a significantly greater manufacturing cost, and so are unacceptable on economic grounds. Others are much more difficult to use as compared to an unprotected needle, and so are rejected by clinicians. Yet further proposals do not allow compliance with best practice protocols.

Most safety devices for use with medical needles employ a sleeve which surrounds the needle to confer protection thereto. Before use, this protects the needle tip from damage as well as guarding against an accidental needle stick injury. After use, the sleeve advantageously is locked in its protecting position so that the needle cannot thereafter be exposed for a second time, so protecting anyone handling the used needle against a needle stick injury. Further, it is most advantageous for a person handling a needle to see whether or not the sleeve has been locked in its protecting position, also indicating that the needle cannot be used, or whether the sleeve is free to expose the needle so that a medical procedure can be performed.

An early example of a safety needle device with an indicator is described in U.S. Pat. No. 5,338,310 (Becton Dickinson). This device has a needle guard which, after use, is manually pushed to and locked at a protecting position. A safety indicator is provided for visually indicating that the needle guard is not locked in the extended position, an obscurement means being provided on the needle guard for obscuring or changing the appearance of, the safety indicator when the needle guard is in the locked extended position.

U.S. Pat. No. 6,547,764 (Novo Nordisk) describes a needle device for a pen injector and which is fitted with a safety shield guided by tracks on the needle hub such that the shield is turned with respect to the hub during use to effect an injection. Different coloured regions on the hub become visible though a transparent area of the shield before and after use. The alignment of the coloured bands with the transparent area is critical to give a correct indication, and the tracks on the hub as well as the followers on the shield must also be aligned properly during manufacture to give a correct indication to an end user.

The safety needle assembly of US2003/0120209 (Novo Nordisk) has a needle shield which slides within a body provided with a window through which a in which a component of the assembly can be seen, to give an indication of whether the assembly is set for use or has been used and the shield is locked in a protecting position. By having a relatively small window in the body of the assembly, it is not immediately apparent to a user on picking up the assembly whether it has been used; and after use, it is not immediately clear that the shield has been locked in its protecting position.

As is apparent from this prior art, there is a significant demand for a protective device for use with a needle which allows a clinician to use the needle in much the same way as is done with an unprotected needle, but which has a sleeve which can be locked in a protecting position and the device gives a clear visual indication that this is so. Though locking of the sleeve could take place without the needle actually being used to perform a medical procedure, for example through mishandling, such a clear visual indication will also show that the needle has been used and so should be discarded safely.

### BRIEF SUMMARY OF THE INVENTION

Bearing in mind the above, this invention provides a safety arrangement for a medical needle having a mount end and a sharp tip, which arrangement comprises:
a safety needle assembly, comprising:
a medical needle having a mount end and a sharp tip;
a support adapted directly or indirectly to carry the mount end of the needle so that the needle projects forwardly away therefrom;
a sleeve mounted directly or indirectly on the support and being slidable with respect thereto from an initial position where the sleeve covers at least the greater part of the needle to a retracted position where the tip of the needle and a part of the needle back from its tip is exposed, and then to a protecting position where the sleeve covers the needle tip and at least part of the needle back from its tip, and wherein at least a part of the sleeve is translucent;
a resilient means for urging the sleeve towards said protecting position;
a blocking means for blocking movement of the sleeve from its protecting position;
a control member for the blocking means to enable movement of the sleeve to the retracted position, the control member being mounted for axial movement within the sleeve such that movement of the sleeve towards the retracted position causes the control member to advance within the sleeve to said translucent part of the sleeve; and
connection means for holding the control member within the sleeve at said translucent part thereof during movement of the sleeve towards the protecting position, so that the control member is maintained at its visible position through the translucent part of the sleeve.

It will be appreciated that the safety arrangement of this invention has a control member for the blocking arrangement which serves to lock the sleeve of the safety arrangement in its protecting position, following use of the needle to perform a medical procedure. The control member is moved deeper into the sleeve in the course of the movement of the sleeve from its initial position to its withdrawn position and then back to a protecting position which may substantially correspond to the initial position, the control member then being visible through the translucent part of the sleeve so as to give the required visual indication that the needle has been used and the sleeve has been locked in its protecting position.

According to a further aspect of this invention, there is provided a safety arrangement in combination with a hypodermic syringe having a cylindrical body, a spigot projecting forwardly from the body for receiving a needle mounting hub, wherein said support includes a socket connected to the spigot of the hypodermic syringe, the support being provided with a needle to project forwardly from the syringe with the needle in communication with the spigot, and the sleeve being slidable on the external surface of the syringe body.

In the alternative, there may be provided a safety arrangement in combination with an injection device adapted to hold a cartridge of medicament, said device having a cylindrical body provided with a spigot at its forward end, wherein said support includes a socket for receiving the spigot of the device and being provided with a needle to project forwardly from the spigot, the needle having a rear end in communication with a cartridge carried by the device, and the support has an external wall on which the sleeve is slidably supported.

### BRIEF DESCRIPTION OF THE SEVERAL VIEWS OF THE DRAWINGS

The drawings show several specific embodiments of safety arrangements for medical needles, constructed and arranged in accordance with this invention, though solely by way of example. In the drawings:

FIGS. 1A to 1E show the first embodiment, having an internal spring, with the sleeve moving from an initial position (FIG. 1A) to a fully withdrawn position (FIG. 1C) and then to a protecting position (FIG. 1E);

FIGS. 1F and 1G are detail views on an enlarged scale of part of the embodiment shown in FIG. 1A;

FIGS. 2A to 2E respectively correspond to FIGS. 1A to 1E, but of the second embodiment, having an external spring;

FIGS. 3A to 3D show a modified form of the second embodiment, also having an external spring but arranged to be resettable, with the sleeve locked in the protecting position (FIG. 3A) and then being reset, ready for re-use (FIG. 3D); and

FIGS. 4A to 4E are similar to FIGS. 2A to 2E but of a third embodiment, having a spring external to the blocking member and being used with a syringe.

### DETAILED DESCRIPTION OF THE INVENTION

Said part of the sleeve through which the control member is visible may be translucent, so long as the control member is sufficiently strongly coloured or is of a high visibility material so as to be visible through the sleeve, or said part of the sleeve may be transparent, in which case there is a lesser need for the control member to be so strongly coloured. Said part may be in the form of a window formed through the sleeve, in which case the control member may easily be seen when in the part of the window. In the alternative, substantially the whole of the sleeve may be translucent or transparent, for example by being moulded from a transparent plastics material.

The movement of the sleeve from its initial position to its withdrawn position and then to its protecting position has to move the control member further into the sleeve to a position where the control member is visible within the sleeve. In preferred embodiments, the control member is pushed forwardly within the sleeve as the sleeve moves rearwardly to expose the needle in the course of performing a medical procedure and the connection means then serves to maintain the control member in that forward position on the return of the sleeve to its protecting position. For this purpose, the sleeve may frictionally engage the control member whereby the control member will remain in its visible position when the sleeve moves back to its protecting position. In the alternative, the connection means may comprise said resilient means acting on the control member in combination with a releasable connection between the control member and the sleeve, said releasable connection acting between the control member and the sleeve when the control member is in an initial position before use of the assembly.

The control member may have a first position where it is not visible and movement of the sleeve from its initial position is permitted, the control member being moved into the sleeve to a visible position by movement of the sleeve from its initial position and back to its protecting position. A further possibility is for the control member to have a first position where a part of the control member is visible and movement of the sleeve from its initial position is permitted, the control member being moved further into the sleeve to a visible position where a greater part of the control member is visible, by movement of the sleeve from its initial position and back to its protecting position.

In a preferred embodiment of the safety arrangement of this invention, the blocking means comprises a blocking member at least a part of which projects forwardly from the support, the blocking member being movable between a non-blocking position where the blocking member extends generally parallel to the needle axis and the sleeve may slide to its retracted position and a blocking position where the blocking member has moved from its non-blocking position so as to be disposed between the support and a part of the sleeve, thereby blocking movement of the sleeve away from its protecting position.

The blocking member may be tubular and when in its non-blocking position is generally coaxial with the sleeve and needle, and when in its blocking position extends at an acute angle to the needle axis.

For this embodiment, the control member when in its first position may be located partially within the sleeve and support the blocking member to lie substantially coaxial with the sleeve. When in its visible position, the control member is located deeper within the sleeve to release the blocking member for movement to its blocking position.

In an arrangement where the initial and protecting positions of the sleeve are the same, and so the needle tip is at all times covered other than when the needle is in a body, there is no possibility of the needle being touched accidentally, either by a clinician or by some other thing. If the sleeve intentionally touches something to an extent sufficient to expose the needle tip, return of the sleeve to its fully forward position will lock the sleeve, so preventing use of the needle to perform an injection.

In the following description of the drawings showing embodiments of this invention, the terms front, forward, and so on are used to refer to that end of the needle assembly whereat the sharp tip of the needle is located and also to the direction of insertion of the needle into a body. Conversely, the terms rear, rearwardly and so on are used to refer to the other end of the needle assembly, to which is connected other equipment such as a syringe or a blood collection system, and also to the direction of removal of a needle from a body.

Further, like components throughout the various embodiments are given like reference characters and will not be described in detail, for each embodiment.

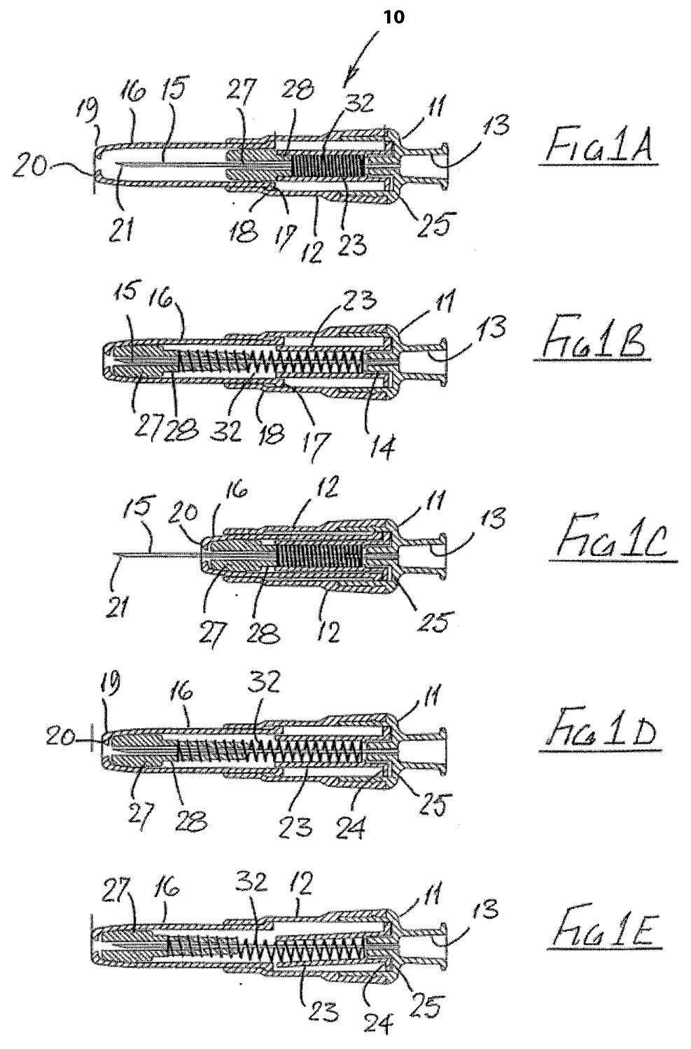

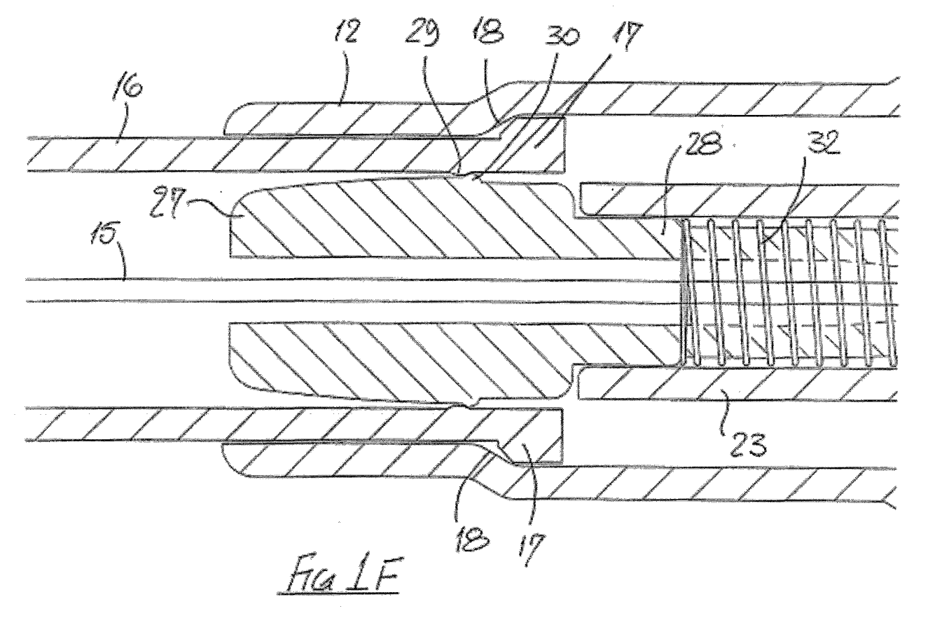

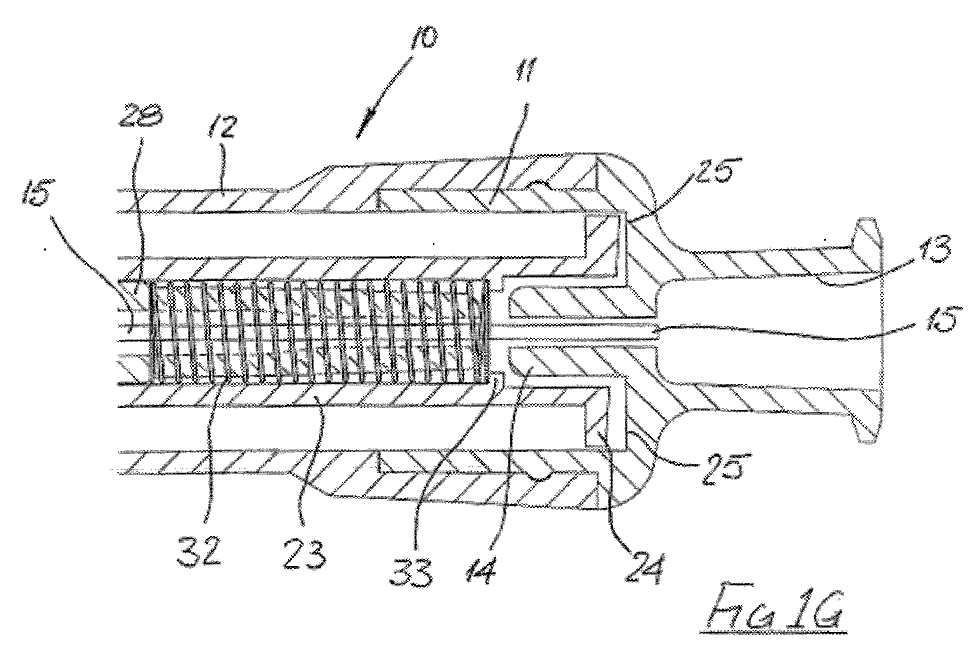

### FIGS. 1A to 1G

The first embodiment of needle assembly of this invention shown in FIGS. 1A to 1G comprises a tubular housing 10 assembled from a rear part 11 and a front part 12, permanently secured together. The rear part 11 includes a tapered socket 13 for receiving the hub of a syringe in the manner of a conventional taper-slip lock, thereby permitting the assembly to be mounted on a syringe ready for use. Internally, the rear part 11 has a boss 14 which carries the mount end of a needle 15, in a manner well known in the art.

The syringe with which the embodiment is to be used may be a pre-filled syringe, such that pre-attachment of the needle assembly immediately renders the syringe ready for use. Rather than the taper slip lock shown, a threaded connector such as a Luer lock may be employed. Alternatively, the rear part may be configured for use with known forms of phlebotomy devices for collecting blood.

Slidably mounted in the forward region of the front part 12 is a tubular sleeve 16, the rear end of the sleeve having an external flange 17 which engages a shoulder 18 formed internally within the front part 12. The sleeve is thus constrained against further forward movement from the position shown in FIGS. 1A and 1E, but may slide rearwardly as shown in FIGS. 1B and 1C. The forward end 19 of the sleeve 16 has an in-turned lip 20 and the sleeve is of a sufficient length such that when its flange 17 engages shoulder 18, the lip 20 is disposed beyond the sharp tip 21 of the needle.

A tubular blocking member 23 surrounds the boss 14 and has a flange 24 at its rear end and disposed adjacent a radial wall 25 of the rear part 11 of the housing. The rearward facing surface of the flange 24 is non-radial with respect to the axis of the blocking member and so that face does not lie flat against the radial wall 25, as shown in FIG. 1G. The blocking member is slightly shorter than the distance between the radial wall 25 and the rear end of the sleeve, when the sleeve is fully forward, as shown in FIG. 1A.

A control member 27 is located within the sleeve 16 and has a rear portion 28 which is receivable within the forward part of the blocking member 23, the control member being profiled to limit rearward movement thereof into the blocking member. A releasable connection is formed between the control member 27 and the internal surface of the sleeve 16, shown in more detail in FIG. 1F, whereby the control member is held against movement forwardly within the sleeve 16 until sufficient force is applied to the sleeve in the rearward direction while the control member 27 is held stationary by abutting the forward end of the blocking member and the flange 24 of the blocking member 23 abuts the rear wall 25 of the housing 10. The releasable connection comprises an internal annular rib 29 engaged with an external preferably segmented annular rib 30 on the control member, or conversely the rib 29 could be segmented and the rib 30 continuous. Sufficient force on the sleeve will break the connection by causing the sleeve rib 29 to ride over the control member rib 30 whereafter the control member may slide freely within the sleeve between the internal face of lip 20 and the sleeve rib 29.

A helical compression spring 32 is disposed within the blocking member 23 and acts between the rear face of the control member 27 and an annular abutment 33 formed within the blocking member, just forward of the boss 14. The force exerted by the spring 32 is insufficient to break the connection between the control member and the sleeve when the assembly is in its initial position as shown in FIG. 1A. The rearward projection of rear portion 28 of the control member 27 is visible in FIG. 1G; this extended projection is intended to support the spring 32 to remain essentially co-axial with the needle.

The operation of the assembly described above will now be described. The initial setting is with the assembly as shown in FIGS. 1A, 1F and 1G with the sleeve 16 fully forward and wholly protecting the needle 15; the control member 27 is connected to the sleeve and is urged forwardly by the spring 32, transferring the spring force to the sleeve. In this position, flange 17 of the sleeve engages shoulder 18 of the front part 12. The rear portion 28 of the control member is located in the blocking member 23, so maintaining that member co-axial with the needle 15.

During initial rearward movement of the sleeve 16, for example by being pressed against a body, the control member is maintained stationary by the blocking member 23, in contact with the rear wall 25. If sufficient force is applied to the sleeve 16 to break the connection, the sleeve will slide on to the blocking member 23. In addition, the control member is released to move forwardly under the action of the spring, until the control member engages the internal face of the sleeve lip 20. The spring thus continues to urge the sleeve forwardly through the control member, but a force applied rearwardly to the sleeve greater than the spring force will allow continued progress of the sleeve, rearwardly.

FIG. 1C shows the sleeve in its extreme rearward position, with the needle 15 projecting to its fullest extent, from the housing 10. Here, the rear portion 28 of the control member has once more entered the blocking member and the flange 17 of the sleeve abuts the flange 24 of the blocking member. Sufficient reduction on the rearward force on the sleeve (for example, by withdrawing the assembly away from a body) will allow the sleeve to move forwards under the action of the spring, as shown in FIG. 1D. On the sleeve moving to its protecting position shown in FIG. 1E, the blocking member 23 is free of the sleeve and so moves to the position shown in that Figure, by virtue of the non-radial face of its flange 24 engaging the radial wall 25 of the housing rear part 11, under the action of spring 32. When in its non-axial position, the blocking member blocks rearward movement of the sleeve away from its protecting position, so rendering safe the needle.

It will be appreciated that in clinical use, as the sleeve 16 comes into contact with the pierceable membrane (e.g. the skin) of a body, the sleeve will automatically move rearwardly from its initial position, allowing penetration of the needle into the body. Further, once the connection between the sleeve and the control member has been broken, the mechanism will automatically lock on the return of the sleeve to the position shown in FIG. 1E. Thus, release of the sleeve from the position shown in FIG. 1B but before the sleeve has moved to the position shown in FIG. 1C will still result in the protecting position of FIG. 1E being achieved.

If the assembly is used in conjunction with a syringe to undertake drug draw-up from a phial or ampoule into the syringe, this particular assembly must be discarded and a second assembly fitted to the syringe, to perform an injection. This is in fact the preferred clinical procedure since a lubricated and uncontaminated new needle should be used for body penetration. As well as protecting the needle, the assembly has the advantage of enforcing the “new-needle” clinical procedure, even should a clinician be disinclined to follow the specified procedure.

The control member 27 is preferably made from a highly visible (e.g. strongly-coloured) plastics material, whereas the sleeve 16 is preferably made of a translucent plastics material. Thus, a simple inspection of the assembly will show whether it has been used, because the control member can be seen at the forward end of the sleeve, or whether it is ready for use, because the control member is not present within that part of the sleeve, beyond the front part 12 of the housing 10 and irrespective of the position of the control member.

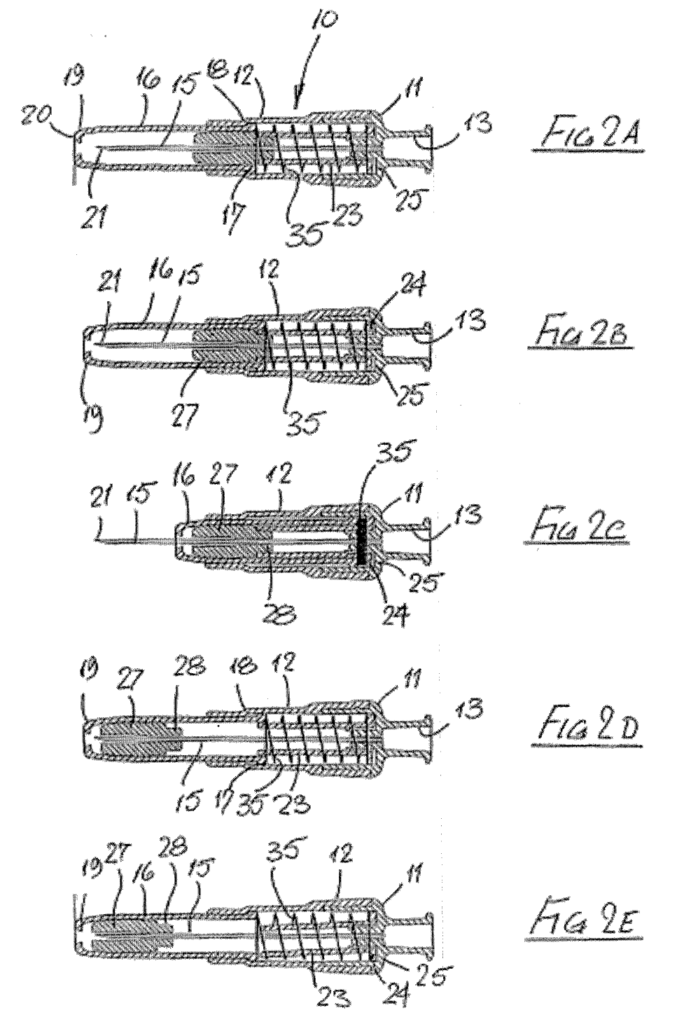

FIGS. 2A to 2E

The second embodiment of FIGS. 2A to 2E is generally similar to that of FIGS. 1A to 1E, except that the connection between the control member 27 and the sleeve 16 is differently configured, and a larger spring 35 is employed, external to the blocking member 23. The spring acts between the flange 24 of the blocking member and the rear face of the sleeve 16, so directly urging the sleeve forwardly, irrespective of the sleeve position with respect to the housing 10.

A simple friction connection is employed between the control member 27 and the sleeve 16, with sufficient friction to ensure the control member remains stationary within the sleeve until sufficient force is applied to the sleeve to overcome that friction. Then, the sleeve will move rearwardly while the control member 27 is held stationary by the blocking member and so is advanced relatively, within the sleeve. Subsequently, on forward movement of the sleeve under the action of the spring 35, the control member moves forward with the sleeve and so comes free of the blocking member. Thereafter, this permits the blocking member to perform its locking action as described with reference to FIG. 1.

In this second embodiment, the amount of rearward movement of the sleeve needed subsequently to result in the disengagement of the blocking member 23 from the control member may be controlled by appropriate selection of the length of the rear portion 28 of the control member 27. With a short rear portion, only small rearward movement of the sleeve will result in an earlier disengagement of the blocking member. Conversely, with a long rear portion, a much greater rearward movement of the sleeve is required before subsequent forward movement of the sleeve disengages the control member from the blocking member.

The action with a long rear portion 28 may be advantageous where the assembly is to be used to perform drug draw-up from a phial or ampoule, before the same assembly is to be used to perform an injection, where procedures permit the same needle to be used for draw-up and subsequent injection into a body—for example with the delivery of insulin. The sleeve may appropriately be marked to show its maximum movement before locking will occur on subsequent release of the sleeve and provided that movement is not exceeded, then the assembly may be used firstly to undertake drug draw-up and secondly to perform an injection, fully inserting the needle to its correct depth, whereafter the assembly will be rendered safe.

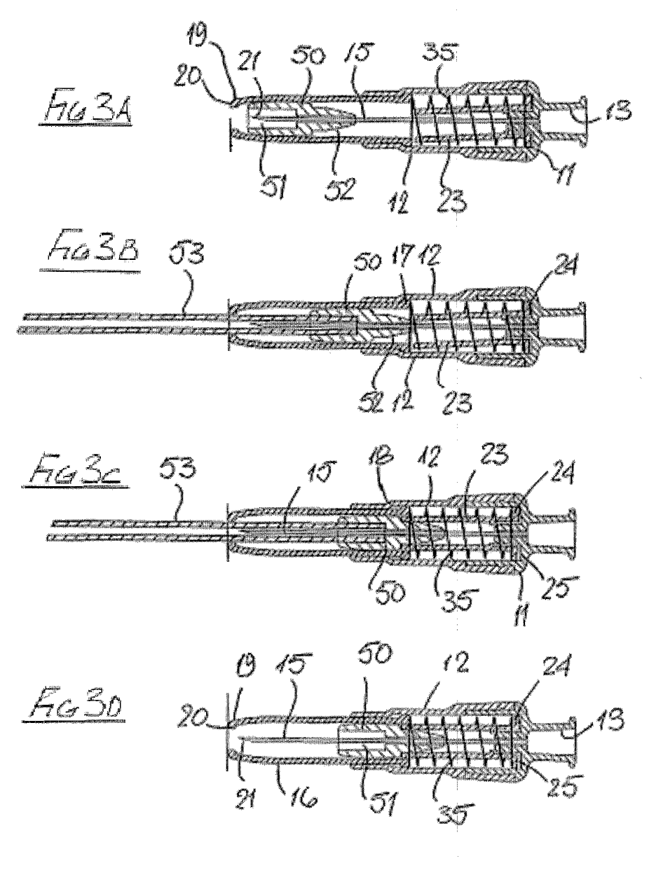

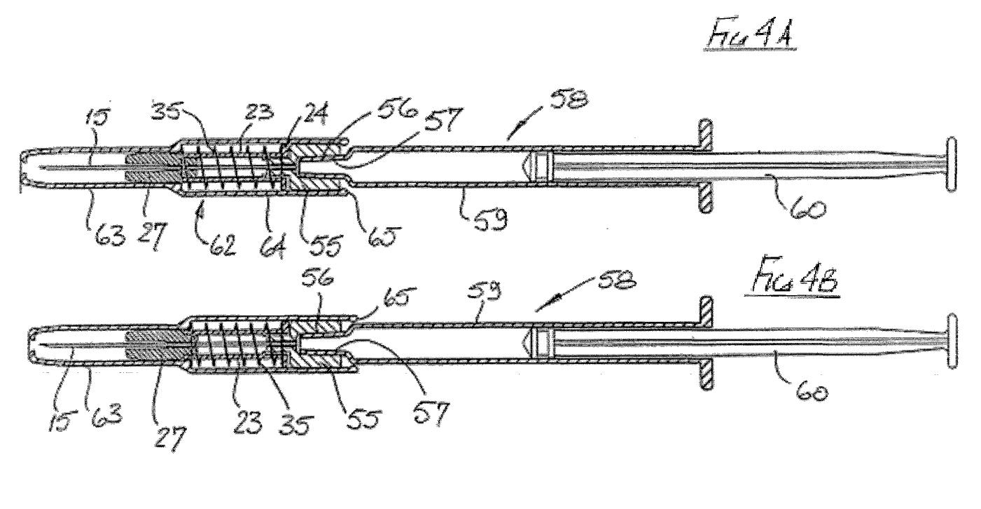

### FIGS. 3A to 3D

This embodiment is a modified form of the second embodiment shown in FIGS. 2A to 2E. The modification is solely to the control member, which is differently profiled as shown in FIG. 3A. The control member 50 is provided with a counter-bore 51 at its forward end and the rear portion 52 tapers towards its free end. Such a control member 50 allows resetting of the assembly to a ready-to-use condition, from a locked condition.

FIG. 3A shows the assembly in its protecting (locked) condition, this corresponding to the setting of FIG. 2E. The mechanism may be reset by means of a tool having a fine tubular shaft 53, receivable through the lip 20 of the sleeve 16 and into the counter-bore 51 of control member 50. Using this tool, the control member 50 may be pushed rearwardly, overcoming the friction between the control member 50 and the sleeve 16, until the control member re-enters the blocking member 23. The tapered profile of the rear portion 52 lifts the control member out of its locking position (FIG. 3B) and continued rearward movement of the control member will bring the blocking member co-axial with the needle, as shown in FIG. 3C. Removal of the shaft 53 leaves the assembly reset, ready for use.

There is no risk to a clinician in using the tool to reset the mechanism. There is no access to the needle tip until the shaft 53 has been used to complete the resetting; during insertion of the shaft, the sleeve protects the needle and no manual access can be gained to the needle tip.

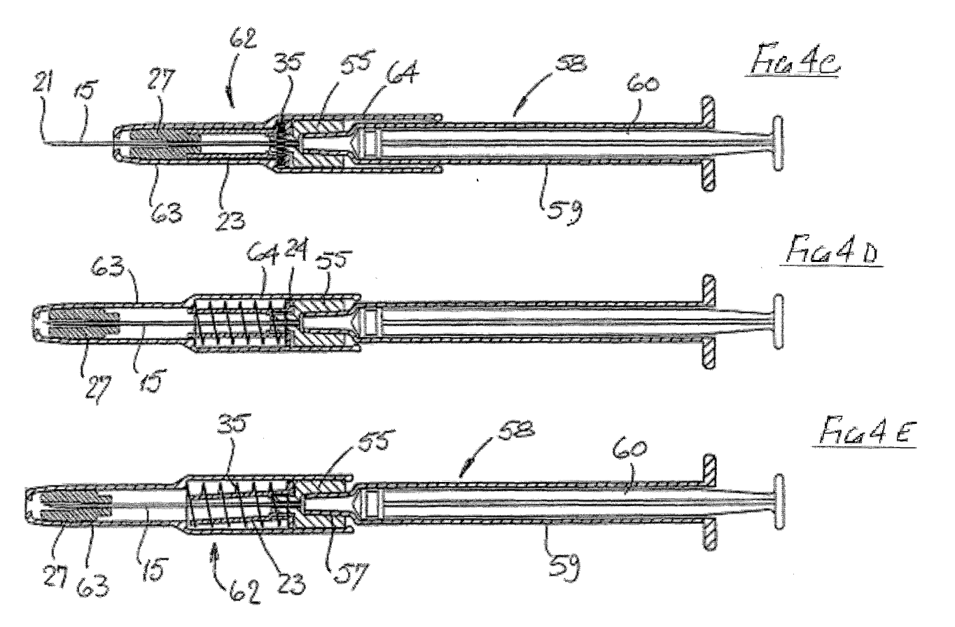

### FIGS. 4A to 4E

A third embodiment of this invention is shown in FIGS. 4A to 4E. This embodiment has a support wall 55 provided with a socket 56 to permit the assembly to be mounted on the hub 57 of a conventional syringe 58, the hub and socket together forming a conventional taper slip lock. A conventional Luer lock could be used, instead. The syringe has a cylindrical body 59 within which is mounted a plunger 60, to permit charging of the syringe and discharging of a drug, through a needle 15 supported on wall 55. A sleeve 62 has a forward portion 63 corresponding to sleeve 16 of the previous embodiments and a rearward portion 64 formed integrally with the forward portion 63. The rearward portion has a sufficient diameter to fit over the cylindrical body 59 of the syringe and is provided with an annular bead 65 at its free end, to stop the sleeve 62 coming off the wall 55.

Internally, the arrangement is essentially the same as that of the embodiment of FIG. 2, and so includes a blocking member 23, a control member 27 and a spring 35 external of the blocking member. The control member 27 is a frictional fit within the forward portion 63 of the sleeve 62 and so may be slid forwardly within the forward portion, as the sleeve 62 is moved rearwardly. As previously, sufficient forward movement of the control member within the forward portion 63 allows the blocking member 23 to move to its inclined position shown in FIG. 4E once the sleeve 62 has moved to its protecting position, so thereafter preventing retracting movement of the sleeve.

In all of the above embodiments, the respective sleeves may be made transparent, translucent or provided with a transparent or translucent window. By manufacturing the respective control member from a strongly-coloured material, the position of the control member within the sleeve will readily be discernible. Then, when the safety device has been used and the control member is fully forward, this will immediately be apparent on looking at the assembly.

I claim:

1. A safety needle assembly, comprising:
a medical needle having a mount end and a sharp tip;
a support adapted directly or indirectly to carry the mount end of the needle so that the needle projects forwardly away therefrom;
a sleeve mounted directly or indirectly on the support and being slidable with respect thereto from an initial position where the sleeve covers at least the greater part of the needle to a retracted position where the tip of the needle and a part of the needle back from its tip is exposed, and then to a protecting position where the sleeve covers the needle tip and at least part of the needle back from its tip, and wherein at least a part of the sleeve is translucent;
a resilient means for urging the sleeve towards said protecting position;
a blocking means for blocking movement of the sleeve from its protecting position;
a control member for the blocking means to enable movement of the sleeve to the retracted position, the control member being mounted for axial movement within the sleeve such that movement of the sleeve towards the retracted position causes the control member to advance within the sleeve to said translucent part of the sleeve; and
connection means for holding the control member within the sleeve at said translucent part thereof during movement of the sleeve towards the protecting position, so that the control member is maintained at its visible position through the translucent part of the sleeve.

2. A safety needle assembly as claimed in claim 1, wherein said part of the sleeve is transparent.

3. A safety needle assembly as claimed in claim 2, wherein said part of the sleeve comprises a window formed therethrough.

4. A safety needle assembly as claimed in claim 1, wherein substantially the whole of the sleeve is translucent.

5. A safety needle assembly as claimed in claim 1, wherein substantially the whole of the sleeve is transparent.

6. A safety needle assembly as claimed in claim 1, wherein the control member is of a strongly-coloured material to enhance the visibility thereof through said part of the sleeve.

7. A safety needle assembly as claimed in claim 1, wherein control member has a first position where the control member is not visible and movement of the sleeve from its initial position is permitted, the control member being moved into the sleeve to a visible position by movement of the sleeve from its initial position and back to its protecting position.

8. A safety needle assembly as claimed in claim 1, wherein the control member has a first position where a part of the control member is visible and movement of the sleeve from its initial position is permitted, the control member being moved further into the sleeve to a visible position where a greater part of the control member is visible, by movement of the sleeve from its initial position and back to its protecting position.

9. A safety needle assembly as claimed in claim 1, wherein the connection means provides frictional engagement between the control member and the sleeve.

10. A safety needle assembly as claimed in claim 1, wherein the connection means comprises said resilient means acting on the control member in combination with a releasable connection between the control member and the sleeve, said releasable connection acting between the control member and the sleeve when the control member is in an initial position before use of the assembly.

11. A safety needle assembly as claimed in claim 1, wherein the blocking means comprises a blocking member at least a part of which projects forwardly from the support, the blocking member being movable between a non-blocking position where the blocking member extends generally parallel to the needle axis and the sleeve may slide to its retracted position and a blocking position where the blocking member has moved from its non-blocking position so as to be disposed between the support and a part of the sleeve, thereby blocking movement of the sleeve away from its protecting position.

12. A safety needle assembly as claimed in claim 11, wherein the blocking member is tubular and when in its non-blocking position is generally co-axial with the sleeve and needle, and when in its blocking position extends at an acute angle to the needle axis.

13. A safety needle assembly as claimed in claim 12, wherein the control member has a first position located partially within the sleeve to support the blocking member to lie substantially coaxial with the sleeve, and a visible position where the control member is located fully within the sleeve to release the blocking member for movement to its blocking position.

14. A safety needle assembly as claimed in claim 1 in combination with a hypodermic syringe having a cylindrical body, a spigot projecting forwardly from the body for receiving a needle mounting hub, wherein said support includes a socket connected to the spigot of the hypodermic syringe, the support being provided with a needle to project forwardly from the syringe with the needle in communication with the spigot, and the sleeve being slidable on the external surface of the syringe body.

15. A safety needle assembly as claimed in claim 1 in combination with an injection device adapted to hold a cartridge of medicament, said device having a cylindrical body provided with a spigot at its forward end, wherein said support includes a socket for receiving the spigot of the device and being provided with a needle to project forwardly from the spigot, the needle having a rear end in communication with a cartridge carried by the device, and the support has an external wall on which the sleeve is slidably supported.
